(19) United States
(12) Patent Application Publication
(10) Pub. No.: US 2007/0238970 A1
(43) Pub. Date: Oct. 11, 2007

(54) Magnetic resonance imaging for interventional procedures

(75) Inventors: Sebastian KOZERKE, Hedingen (CH); Reza RAZAVI, London (GB); Derek Lionel Glendon HILL, London (GB); Sanjeet Rajivlochan HEGDE, Beckenham (GB)

Correspondence Address: PHILIPS INTELLECTUAL PROPERTY & STANDARDS, 595 MINER ROAD, CLEVELAND, OH, 44143, US

(73) Assignees: KONINKLIJKE PHILIPS ELECTRONICS N.V., Eindhoven (NL); KING'S COLLEGE LONDON, London Greater (GB)

(21) Appl. No.: 11/568,323

(22) PCT Filed: Apr. 28, 2005
(86) PCT No.: PCT/IB05/51383

§ 371 Date: Oct. 26, 2006

(30) Foreign Application Priority Data
Apr. 29, 2004 (EP) 04252513.9

(51) Int. Cl. A61B 5/05

(52) U.S. Cl. 600/410

(57) ABSTRACT

Two different RF-frequency ranges or bands are employed viz. at the localisation RF-frequency and at the imaging RF-frequency, respectively. At these respective RF-frequency ranges different types of magnetic resonance signals are acquired. At the localisation RF-frequency a high sensitivity for the position of the interventional device is achieved. At the imaging RF-frequency a high sensitivity for image information, i.e. contrast resolution, of the anatomical structures of the patient to be examined is achieved.

The invention relates to an magnetic resonance imaging method which localizes an interventional device.

Such an magnetic resonance imaging method is known from the U.S. Pat. No. 6,574,497.

In the known magnetic resonance imaging method compounds containing ¹⁹F material are used as a contrast agent in interventional magnetic resonance angiography. The known method makes use of the circumstance that ¹⁹F has a reasonable sensitivity compared to protons at the RF-frequency range employed in current MR scanners. The lumen of the interventional device is filled with the ¹⁹F contrast agent. The magnetic resonance image that is reconstructed from the magnetic resonance signals acquired at the conventional RF-frequency range displays the interventional device relative to the anatomy of the patient when the interventional device is introduced in the patient's body. Accordingly, the position of the interventional device is found from the magnetic resonance image, that is the interventional device is localised within the patient's body.

An object of the invention is to provide an magnetic resonance imaging method which more accurately localises the interventional device.

This object is achieved by an magnetic resonance imaging method of the invention wherein

localisation magnetic resonance signals are acquired which represent the actual position of at least a pre-selected portion of an interventional device,

the localisation magnetic resonance signals being acquired at a localisation RF-frequency range

imaging magnetic resonance signals are acquired which represent image information

the imaging magnetic resonance signals being acquired at an imaging RF-frequency range.

The present invention employs two different RF-frequency ranges or bands, viz. at the localisation RF-frequency and at the imaging RF-frequency, respectively. At these respective RF-frequency ranges different types of magnetic resonance signals are acquired. At the localisation RF-frequency a high sensitivity for the position of the interventional device is achieved. At the imaging RF-frequency a high sensitivity for image information, i.e. contrast resolution, of the anatomical structures of the patient to be examined is achieved. That is, by employing separate RF-frequency bands for the localisation and imaging respectively, the acquisition of magnetic resonance signals for localisation and for imaging respectively are independently optimised. The localisation magnetic resonance signals at the localisation RF-frequency include information on the position of the interventional device. The imaging magnetic resonance signals include image information of the object into which the interventional device is introduced. The object is notably a patient to be examined. Hence, on the basis of the localisation magnetic resonance signals and the imaging magnetic resonance signals the actual position of at least the pre-selected portion of the interventional device is established relative to the object, notably the patient's anatomy. These and other aspects of the invention will be further elaborated with reference to the embodiments defined in the dependent Claims.

The localisation magnetic resonance signals and the imaging magnetic resonance signals are spatially encoded by way of magnetic gradient fields that define a common frame of reference. Thus, the localisation magnetic resonance signals represent the position of the pre-selected portion of the interventional device in the frame of reference that is in common with the magnetic resonance image reconstructed from the imaging magnetic resonance signals. Accordingly, the position of the pre-selected portion can be accurately shown in the magnetic resonance image. The pre-selected portion notably has a high sensitivity for MR-excitation at the localisation RF-frequency. This is notably achieved in that the pre-selected portion contains a compound including a nucleus that has its precession (Larmor) frequency in the of the localisation RF-frequency range. Hence, the localisation magnetic resonance signals have a high signal level that is easily and accurately detected. Magnetic resonance acquisition sequences to localise the tip of a catheter that operate at the proton frequency band are known per se from the European patent application EP 0 731 362 and from the international application WO01/73460.

The invention may be employed in a local mode where the localisation magnetic resonance signals pertain to a pre-selected portion of the interventional device. A particular example of the pre-selected portion is notably the distal end of a catheter. For example an expandable balloon is often mounted at the distal end of the catheter. According to one aspect of the invention an amount of a localisation compound, such as a ¹⁹F-compound, is contained in the balloon. The invention may also be employed in a global mode where the localisation magnetic resonance signals pertain to a large portion, —e.g. essentially the most of—the interventional device. This is for example achieved in that the interventional device includes a lumen, or several lumen compartments that extend along the length of the interventional device. This lumen or lumen compartments may be filled with the localisation compound.

According to a further aspect of the invention a magnetic resonance image is reconstructed from both the localisation magnetic resonance signals as well as from the imaging magnetic resonance signals. This reconstructed magnetic resonance image shows the interventional device, or at least its pre-selected portion, within the anatomical surrounding that is represented by the imaging magnetic resonance signals.

Suitable materials for the localisation compound are ¹⁹F-compounds, such as C¹⁹F₃-compounds. A good example appears to be perfluorooctylbromide (C₈¹⁹FBr)(PFOB) These ¹⁹F-compounds have a high sensitivity for selective excitation in the RF-frequency range given by ωₒ=γ|B₀|, where ω₀ is the central frequency in the RF-frequency range, γ is the relevant gyromagnetic ratio, e.g. when a ¹⁹F compound is used, γ=40.06 MHz/T and B₀ is the field strength of the main magnetic field. Good results are achieved when the localisation magnetic resonance signals are acquired at a bandwidth of 1.984 MHz. At this bandwidth—C¹⁹F₃ resonances are selectively excited and in particular perturbation due to signal from C¹⁹F₂ groups are avoided.

According to one aspect of the invention the localization magnetic resonance signals are acquired while one or several magnetic read gradient fields in respective—notably orthogonal—directions, are successively activated. These magnetic read gradient provide sufficient spatial encoding of the localization magnetic resonance signals to establish the position of notably the pre-selected portion of the interventional device. Notably, there is no specific need to acquire localization magnetic resonance signals that enable to fully image the pre-selected portion of the interventional device. Hence, the localization magnetic resonance signals may be non-phase-encoded. This acquisition scheme enables a very rapid acquisition of the localization magnetic resonance signals. Further, this acquisition scheme is operated in the localization RF-frequency band.

The invention also relates to an magnetic resonance imaging system as defined in claim 8. The magnetic resonance imaging system of the invention enables to carry out the magnetic resonance imaging method of the invention and hence to accurately localise the interventional device within the patient's body. The invention further relates to a computer programme as defined in claim 9. The computer programme can be loaded into the working memory of the processor of am magnetic resonance imaging system to enable the magnetic resonance imaging system to carry out the magnetic resonance imaging method of the invention which accurately localises the interventional device within the patient's body. The computer programme of the invention can be supplied on a data carrier such as a CD-rom. Alternatively, the computer programme of the invention can be supplied in the form of e.g. digital, datasets that can be downloaded from a data network such as the world-wide web.

Further, the invention pertains to an interventional device as defined in claim 7. The interventional device of the invention comprises a pre-selected portion that functions as a reservoir to contain a ¹⁹F-compound, such as a C¹⁹F-compound. The interventional device is notably suitable to be localised by way of the magnetic resonance imaging method of the invention.

These and other aspects of the invention will be elucidated with reference to the embodiments described hereinafter and with reference to the accompanying drawing wherein

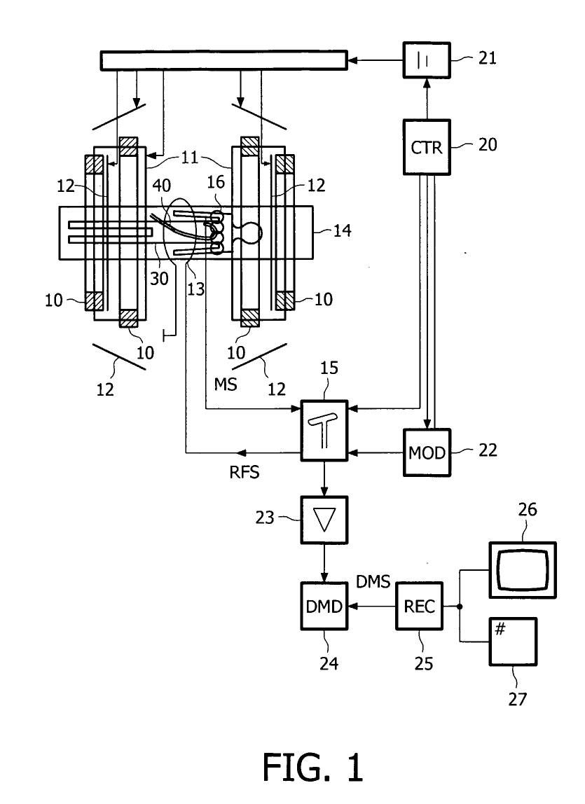

FIG. 1 shows diagrammatically a magnetic resonance imaging system in which the invention is used.

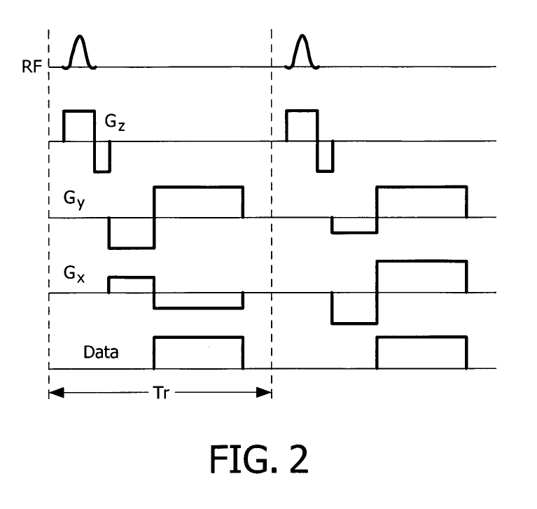

FIG. 2, 3 and 4 show a graphical representations of a magnetic resonance acquisition sequences for the magnetic resonance imaging method of the invention.

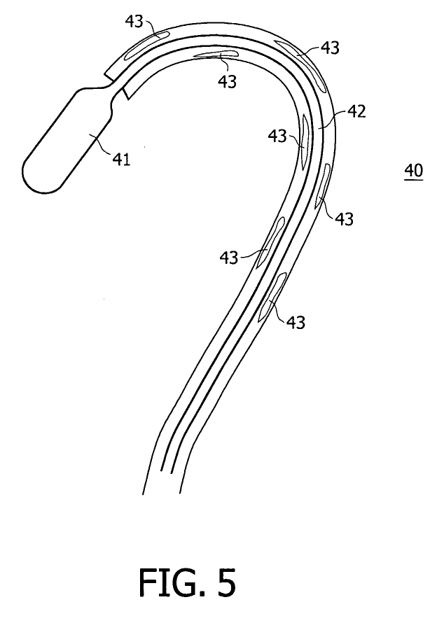

FIG. 5 shows a schematic representation of the interventional device of the invention.

FIG. 1 shows diagrammatically a magnetic resonance imaging system in which the invention is used. The magnetic resonance imaging system includes a set of main coils 10 whereby the steady, uniform magnetic field is generated. The main coils are constructed, for example in such a manner that they enclose a tunnel-shaped examination space. The patient to be examined is placed on a patient carrier which is slid into this tunnel-shaped examination space. The magnetic resonance imaging system also includes a number of gradient coils 11, 12 whereby magnetic fields exhibiting spatial variations, notably in the form of temporary gradients in individual directions, are generated so as to be superposed on the uniform magnetic field. The gradient coils 11, 12 are connected to a controllable power supply unit 21. the gradient coils 11, 12 are energised by application of an electric current by means of the power supply unit 21. The strength, direction and duration of the gradients are controlled by control of the power supply unit. The magnetic resonance imaging system also includes transmission and receiving coils 13, 16 for generating the RF excitation pulses and for picking up the magnetic resonance signals, respectively. The transmission coil 13 is preferably constructed as a body coil 13 whereby (a part of) the object to be examined can be enclosed. The body coil is usually arranged in the magnetic resonance imaging system in such a manner that the patient 30 to be examined is enclosed by the body coil 13 when he or she is arranged in the magnetic resonance imaging system. The body coil 13 acts as a transmission antenna for the transmission of the RF excitation pulses and RF refocusing pulses. Preferably, the body coil 13 involves a spatially uniform intensity distribution of the transmitted RF pulses (RFS). The same coil or antenna is usually used alternately as the transmission coil and the receiving coil. Furthermore, the transmission and receiving coil is usually shaped as a coil, but other geometries where the transmission and receiving coil acts as a transmission and receiving antenna for RF electromagnetic signals are also feasible. The transmission and receiving coil 13 is connected to an electronic transmission and receiving circuit 15.

It is to be noted that it is alternatively possible to use separate receiving and/or transmission coils 16. For example, surface coils 16 can be used as receiving and/or transmission coils. Such surface coils have a high sensitivity in a comparatively small volume. The receiving coils, such as the surface coils, are connected to a demodulator 24 and the received magnetic resonance signals (MS) are demodulated by means of the demodulator 24. The demodulated magnetic resonance signals (DMS) are applied to a reconstruction unit. The receiving coil is connected to a preamplifier 23. The preamplifier 23 amplifies the RF resonance signal (MS) received by the receiving coil 16 and the amplified RF resonance signal is applied to a demodulator 24. The demodulator 24 demodulates the amplified RF resonance signal. The demodulated resonance signal contains the actual information concerning the local spin densities in the part of the object to be imaged. Furthermore, the transmission and receiving circuit 15 is connected to a modulator 22. The modulator 22 and the transmission and receiving circuit 15 activate the transmission coil 13 so as to transmit the RF excitation and refocusing pulses. The reconstruction unit derives one or more image signals from the demodulated magnetic resonance signals (DMS), which image signals represent the image information of the imaged part of the object to be examined. The reconstruction unit 25 in practice is constructed preferably as a digital image processing unit 25 which is programmed so as to derive from the demodulated magnetic resonance signals the image signals which represent the image information of the part of the object to be imaged. The signal on the output of the reconstruction monitor 26, so that the monitor can display the magnetic resonance image. It is alternatively possible to store the signal from the reconstruction unit 25 in a buffer unit 27 while awaiting further processing.

The magnetic resonance imaging system according to the invention is also provided with a control unit 20, for example in the form of a computer which includes a (micro)processor. The control unit 20 controls the execution of the RF excitations and the application of the temporary gradient fields. To this end, the computer program according to the invention is loaded, for example, into the control unit 20 and the reconstruction unit 25.

Notably, the control unit is arranged, e.g. by way of the computer programme of the invention, to enable acquiring magnetic resonance signals in the localisation RF-frequency and in the imaging RF-frequency ranges. To this end the control unit controls the transmission and receiving circuit 15 to operate in these respective frequency ranges. Also the reconstruction unit 25 is arranged to reconstruct the magnetic resonance image from the magnetic resonance signals from both RF-frequency ranges to provide the magnetic resonance image that accurately localises the interventional device 40.

FIG. 2 shows a graphical representation of a magnetic resonance acquisition sequence for the magnetic resonance imaging method of the invention. FIG. 2 shows time lines for the various RF-pulses and temporary magnetic gradient fields (gradient pulses). The sequence has a repetition time T_{R}, the time internal shown between the dashed lines. The sequence comprises a spatially selective RF-excitation that is accompanied by a slice selection gradient (Gz) in the z-direction. Following the RF-excitation further read gradient pulses (Gx, Gy) are applied in directions orthogonal to the slice selection gradient. Magnetic resonance signals are read out during these read gradient pulses. To enable visualisation of the interventional device, the magnetic resonance acquisition sequence is optimised to operate in the localisation FR-frequency range.

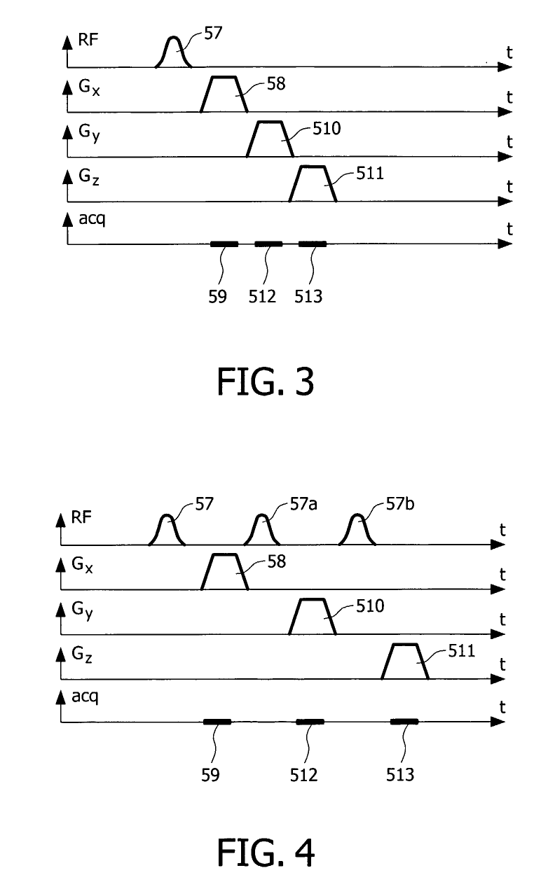

As an alternative to localise the interventional device, interleaved projections using a ¹⁹F steady state free precession (SSFP) sequence are employed. FIG. 3 shows a graphical representation of a magnetic resonance acquisition sequence for the magnetic resonance imaging method of the invention. The diagram shown in FIG. 3 illustrates the execution in time of the sequence in accordance with the invention for the localization of the microcoil provided on an interventional instrument. The upper line shows that the sequence commences with an RF pulse 57 which is not selective, so that magnetization is excited in the entire examination zone. The RF pulse is succeeded by a first gradient pulse 58 which is shown on the next line. The diagrams of the second, the third and the fourth line represent the current through various gradient coils as a function of time. The first gradient pulse 8 concerns a gradient that is applied in the x direction and ensures that the nuclear magnetization in the vicinity of the microcoil performs a processional motion at a frequency which is directly proportional to the corresponding x co-ordinate. The associated magnetic resonance signal that is induced in the microcoil is then collected for the duration of the first gradient pulse 58. The time intervals in which the data acquisition takes place are shown on the last line of the diagram. The data acquisition for the determination of the x co-ordinate of the microcoil thus takes place in a time interval 9. The x gradient pulse is succeeded by a y gradient 510 and a z gradient 511 which are associated with the time intervals 512 and 513 for data acquisition. During the time intervals 59, 512 and 513 the signal has frequencies wherefrom the x, y and z co-ordinates of the microcoil can be derived directly, for example, by Fourier transformation. The position of the interventional instrument whereto the microcoil is attached is thus completely determined.

FIG. 4 shows a graphical representation of another magnetic resonance acquisition sequence for the magnetic resonance imaging method of the invention. The alternative sequence as shown in FIG. 4 comprises two further RF pulses 57a and 57b which are irradiated between the data acquisition intervals 59, 512, and 513 respectively. The RF pulses 57a and 57b serve as refocusing pulses in order to create echo signals for data acquisition with an optimal signal to noise ratio. This makes the method of the invention applicable even if the magnetic resonance signal dephases rapidly due to strong gradients, which can be applied to obtain a high spatial resolution during the localization of the microcoil.

FIG. 5 shows a schematic representation of the interventional device of the invention. The interventional device shown in FIG. 3 has the form of a catheter 40. At the distal end an inflatable balloon 41 is provided. The inflatable balloon is filled with the ¹⁹F-compound. Also the lumen 42 of the catheter 40 is filled with the ¹⁹F-compound. The balloon filled with the ¹⁹F-compound is easily localised by the magnetic resonance imaging method of the inventions. Accordingly, the pre-determined portion formed by the balloon 41 at the distal end is accurately localised. Localisation of the catheter along its length is facilitated by filling the lumen with the ¹⁹F compound. Further, separate reservoirs 43 containing the ¹⁹F-compound are provided along the length of the catheter. The use of the separate reservoirs to contain the ¹⁹F-compound enables continuous localisation of the catheter without the need to fill the lumen with the ¹⁹F-compound so that the lumen may be employed for other functions.

1. A magnetic resonance imaging method wherein
localisation magnetic resonance signals are acquired which represent the actual position of at least a pre-selected portion of an interventional device,
the localisation magnetic resonance signals being acquired at a localisation RF-frequency range
imaging magnetic resonance signals are acquired which represent image information
the imaging magnetic resonance signals being acquired at an imaging RF-frequency range.

2. A magnetic resonance imaging method as claimed in claim 1, wherein during the localization magnetic resonance signals magnetic read gradient fields are applied and the localization magnetic resonance signals are non-phase encoded.

3. A magnetic resonance imaging method as claim in claim 1, wherein
a static magnetic field is applied at a pre-set magnetic field strength,
the imaging RF-frequency is in the range of precession (Larmor) frequencies of imaging nuclei, in particular protons, at the pre-set magnetic field strength and
the localisation RF-frequency range is in the range of precession (Larmor) frequencies of localisation nuclei, in particular not including protons.

4. A magnetic resonance imaging method wherein a magnetic resonance image is reconstructed from the imaging magnetic resonance signals and the localization magnetic resonance signals.

5. A magnetic resonance imaging method as claimed in claim 1, wherein the pre-selected portion of the interventional device contains a localization compound which contains localization protons.

6. A magnetic resonance imaging method as claim in claim 5, wherein the localisation compound contains a ¹⁹F-compound, in particular a C¹⁹F₃-compound.

7. An interventional device comprising a pre-selected portion that is arranged to receive a localisation compound that in particular contains a ¹⁹F-compound, in particular a C¹⁹F₃-compound.

8. An magnetic resonance imaging system comprising
an RF-excitation system
an receiver antennae system to receive magnetic resonance signals
a control unit the control the RF-excitation system and the receiver antennae system
the RF-excitation and the receiver antennae system having adjustable operating RF-frequency bands including a localisation RF-frequency range and an imaging RF-frequency range
the control unit being arranged to
control operating RF-frequency bands the RF-excitation system and of the receiver antennae system and to
acquire localisation magnetic resonance signals at the localisation RF-frequency range acquire imaging magnetic resonance signals at the imaging RF-frequency range.

9. A computer programme comprising instruction to
acquire localisation magnetic resonance signals which represent the actual position of at least a pre-selected portion of an interventional device,
the localisation magnetic resonance signals being acquired at a localisation RF-frequency range
acquire imaging magnetic resonance signals which represent image information
the imaging magnetic resonance signals being acquired at an imaging RF-frequency range.
